(19) United States
(12) Patent Application Publication
(10) Pub. No.: US 2003/0181792 A1
(43) Pub. Date: Sep. 25, 2003

(54) Healthcare outcome measurement, analysis and improvement system

(76) Inventor: Robert Seyko, Mount Clemens, MI (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/347,818

(22) Filed: Jan. 21, 2003

Related U.S. Application Data
(60) Provisional application No. 60/349,831, filed on Jan. 18, 2002.

(51) Int. Cl. A61B 5/00

(52) U.S. Cl. 600/300

(57) ABSTRACT

An automated system for assisting in the treatment of patients using evidence-based medicine. The system includes a patient interface to which information is input by a practitioner. The information is stored in a database. A template is provided based on the diagnosis including suggested orders and pertinent articles for use in providing treatment. The system includes a data warehouse which receives all of the data. Analysis is made of the individual patient data as well as research data to determine the most effective treatments. The templates are updated to provide the most effective treatments.

### RELATED APPLICATION

This application claims priority of U.S. Provisional Patent Application Serial No. 60/349,831 filed Jan. 18, 2002, which is incorporated herein by reference.

### BACKGROUND OF THE INVENTION

Despite the wealth of available medical information, selection of a particular treatment methodology is oftentimes a highly subjective and unstandardized process. As a result, patients having exactly the same disease might be receiving entirely different treatments even though they are being treated in the same medical facility. Therefore, it is conceivable that at least one patient might not be obtaining the most effective care just because that patient's doctor was not aware of new or more effective treatment advances.

In order to overcome this clearly undesirable problem, it would be beneficial if medical professionals had at their disposal a system which monitored and analyzed the outcome of treatment.

### SUMMARY OF THE INVENTION

The system includes a central computer which communicates with a practitioner's terminal. An interface is provided for input of patient data and diagnosis. The system also includes a treatment template for each diagnosis. The template provides evidence-based validated treatment methods from a data warehouse. The template delivers orders and prompts the practitioner for information. The information is stored and analyzed for effectiveness. The data warehouse is updated based on the analysis.

The system structures the data using unique methodology to allow for rapid statistical analysis. The system allows for derivation of optimal practice patterns through the study of this data, and the system reintroduces the results of these studies in combination with filtered existing medical references by offering suggestions for care that define optimal practice patterns real time for use by the physician at bedside. The system therefore achieves continued increasing standardization of patient care and permits rapid analysis of the effectiveness of particular treatment methods. The process is continuous; new therapies, medications and testing will alter the data again allowing for determination of best practice methodology.

### BRIEF DESCRIPTION OF THE DRAWINGS

FIG. 1 is a schematic of a healthcare system in accordance with the invention; and

FIG. 2 is flowchart showing a methodology of use of the system in accordance with the invention.

### DETAILED DESCRIPTION OF THE INVENTION

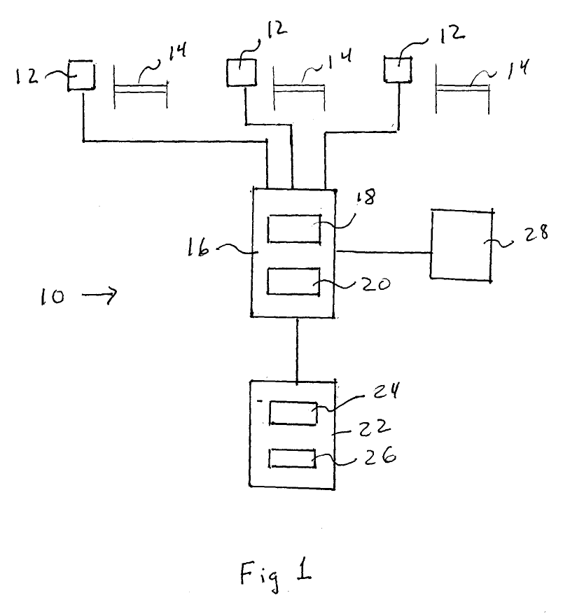

As shown in FIG. 1, the healthcare outcome measurement and analysis system 10 of the present invention tracks, monitors and analyzes the treatment of patients. The system 10 includes a plurality of computer terminals 12 accessible at respective patients' beds 14 which are connected to a central computer 16. The terminal 12 is provided for inputting patient information to the central computer 16 and to provide the practitioner with information regarding effective methods of treatment. The system 10 is preferably adapted to provide individual practitioners with means of obtaining information related to effective methods of care and treatment in regard to a particular diagnosis. Thus, the system 10 can be accessed and directly updated through a terminal 12 wirelessly connected to allow for use at the patient's bedside 14. The central computer 14 has a memory device 18 for storing the patient information and has an interface 20 which is displayed on terminal 12 to prompt the practitioner to enter data.

The interface 20 is used for patient information collection, assisting in medical diagnosis, tracking treatment and outcome and providing resources. The interface 20 prompts the practitioner to enter specific patient information. The patient information includes a medical history, physical exam information, system review information, patient response to treatment, and daily hospital records.

The central computer 14 is electronically connected to a data warehouse 22 for complex analysis of specific clinical data utilizing evidence-based medical literature methodologies. The data warehouse 22 includes a template 24 for each diagnosis. Each template has the most effective treatment protocol, drug or other medical benefit for use in combating a particular condition giving rise to the diagnosis. A library 26 having medical references is indexed for conditions, diseases, or disease combinations in the data warehouse 22. The index and pertinent references are delivered through the template 24 and interface 18 to the practitioner through terminal 12. The data and each literature reference is reviewed (i.e., filtered) to ensure that the protocol or treatment that it recommends is effective, with a particular emphasis being placed on references that teach the best and more successful medical methods. The template 24 also includes orders for gathering information and provides guidance for treatment. It will be appreciated that the database and data warehouse can also be stored on a single computer system or in separate computers. The central computer may be linked to administrative database 28 for use in billing and record keeping.

The methodology for designing a template 24 for particular conditions starts with defining a diagnosis. The methodology will be discussed in using a diagnosis of bronchitis as an example. However, templates can be developed for any condition. The methodology begins with defining outcome measures for the condition, which in the case of bronchitis may be: cough reduction, fever normalization, wheezing reduction, dyspnea reduction, duration of illness reduction, bronchodilator use avoidance/reduction, and corticosteroid use avoidance/reduction. The next step is to review the pertinent evidence-based medical literature. Specific articles will be reviewed and filtered as discussed below. After reviewing the literature, questions are generated regarding treatments which require input regarding the individual patient condition such as “Are antibiotics effective in the treatment of acute bronchitis?”, “Are bronchodilators effective in the treatment of bronchitis?”, “When should bronchodilators be used in the treatment of bronchitis?”, etc. Specific data is defined which is necessary to answer the questions, i.e. presence, duration, severity of the cough; presence, duration, severity of the fever; presence, duration, severity of the wheezing; presence, duration, severity of the dyspnea, hypoxia, etc. The literature is reviewed to make sure that the questions are adequate and then a set of questions are loaded into the template to be presented through the interface 20 to the practitioner are developed. For bronchitis, these questions may include patient assessment of severity of cough, measure of temperature on admission, measure of temperature every 24 hours, FIO2 on admission, daily if SAT is less than 92% PEF on admission, daily if less than 300 l/m, use of antibiotic type and duration, use of bronchodilator type dosing duration, presence/absence of wheezing on physical exam, severity of illness, assessment by practitioner on admission, bacteriologic data from sputum culture, etc. Specific orders are provided to gather the data such as vital signs, WC on admission, daily patient assessment of progress, sputum culture and sensitivity, CRX AP PORT if bedridden, etc.

As treatment progresses, all of the data is entered into the patient's file through the interface and then forwarded to the data warehouse 22 for study. The data is analyzed to determine the best practice methodologies and new order sets are written based on previous analysis. The cycle repeats itself to better treatment. That analysis can be seen in response to stroke management for patients with cerebrovascular accidents. The data is filtered through a number of filters as set forth in the table below.

Example question for stroke management with logic for AWARE software:

In patients with a diagnosis of cerebrovascular accident meeting the following criteria:

a. High probability of disease

b. Age<55 years

c. No thrombotic risk factors (where thrombotic risk is defined as presence of hypertension, diabetes, hyperlipidemia, cigarette smoking, and/or strong family history of cerebrovascular accident)

d. Presence of hypercoaguable state

were treatment and outcomes affected by the presence of a measurable coagulation abnormality?

Filter 1:

What is the total number of patients who presented with a diagnosis of CVA in the inpatient setting?

Filter 2:

What percentage of these patients had a diagnosis of non-hemorrhagic stroke?

Filter 3:

Of these patients with a diagnosis of non-hemorrhagic CVA, what percentage was felt to have a high probability of disease (defined as>75% likelihood of disease)?

Filter 4:

What percentage of these patients were<55 years in age?

Filter 5:

What percentage of these patients had no thrombotic risk factors (where thrombotic risk factors are defined as presence of hypertension, diabetes, hyperlipidemia, cigarette smoking, and/or family history of CVA).

Filter 6:

What percentage of these patients had a demonstrable measure of hypercoaguability (where hypercoaguability is defined as abnormality in any of the following parameters):

Homocysteine level

Anticardiolipin antibodies

Thrombocytosis (platelet count>1,000,000)

Filter 7:

In the subgroup of patients thus identified, what are the outcome variances among patients with a coagulation parameter abnormality in the following areas:

Mortality rate

Daily rate of functional motor change

Discharge functional level (relative to admission)

Filter 8:

What are the treatment variables among those patients with CVA and an abnormal coagulation parameter?

Filter 9:

Were there differences in the measured outcomes relative to the treatment variables identified above?

Filter 10:

Which treatment yielded “best practice” results?

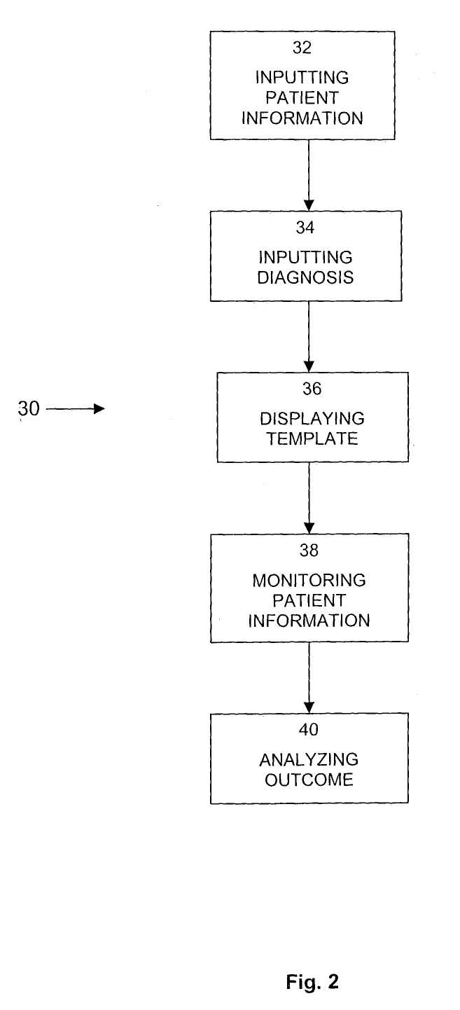

As shown in FIG. 2, a method of use 30 for treating a patient utilizing the system 10 includes inputting 32 the patient's medical information, including any recent physical exam, into the computer 16 through the interface 20. The interface prompts the practitioner to enter the patient's history, physical exam information and history of present condition and review of systems into the database. The practitioner then enters 34 a working diagnosis through the interface. The computer selects and displays 36 a template 24 for the diagnosis and activates orders. The template 24 includes orders that will carry throughout the patient's stay, current state of the art literature references related to the condition, and treatment. The practitioner will then review the current evidence-based medicine as prompted by the working diagnosis and enter orders guided by the data analysis.

This clinical information will be derived from two main sources: 1) data derived from use of the software itself (that is, actual patient data as entered by the practitioner using the system and subsequently analyzed) and 2) pertinent evidence-based medical literature relating to the treatment of the diagnosed disease. As discussed below, this information is filtered and reviewed by an administrator via statistical methods such that the practitioner is provided with reliable and effective treatment information for the diagnosed disease.

Based upon the review of the information thus learned through real time direct patient care outcomes in combination with filtered literature data, applicable order suggestions for treatment are generated and transmitted to the practitioner user computer interface for potential execution by medical personnel. The practitioner user will retain the ability to either accept the suggestions offered or utilize other treatment methodologies of his/her own choice. The variances in approaches taken by the practitioner users will allow for further study of patient outcomes.

The system records 36 the practitioner order selection, prints orders and/or transfers the orders to a hospital computer 28 for processing. After the initial orders are obtained, the practitioner reviews the necessity for subsequent studies. As the treatment progresses, the history and physical material is electronically transferred to the patient's medical record in the hospital computer 28. This includes the practitioner's assessment (working diagnosis) and the treatment plan which includes protocol and orders. As the patient's stay continues, information on the patient's condition is entered 38 into the system 10 on a periodic basis. Any problem variances that develop are noted. Variances may include conditions such as fever, elevated blood sugar, etc. When a variance occurs, the system will prompt the practitioner for specific clinical and supportive data. The system then from the data warehouse 22 provides subprotocol solutions to address the variances. The practitioner will continue to provide information on patient outcome. This information will be entered into the system and the system will continuously analyze 40 the data in real time and prompt the practitioner for data necessary to derive outcome assessments. Once the clinical condition has been resolved, the system gathers selected data to serve as a discharge summary and final data is compiled for study and analysis. Study and analysis is conducted on an evidence-based system to derive optimal patient management for subsequent encounters.

Therefore, it can be seen that the use of the system 10 and method 30 of the present invention can be used to continuously improve medical care by permitting practitioners to be universally aware (preferably while at a patient's bedside making decisions as to possible treatment protocols) of the most effective treatments as determined by the software for a particular diagnosis. Furthermore, since the data warehouse 22 is continuously updated by the addition of information entered in the care of their patients and those of all practitioners using the system in conjunction with carefully filtered medical references, practitioners can be automatically universally aware of new treatment strategies once they are proven to be safe and effective.

By study of larger groups of patients exposed to particular treatments, medical professionals can obtain evidence regarding the success or failure of a particular treatment much faster than would be allowed by modern methods. To this end, it is also understood that the system is capable of determining value of a treatment or drug (where value is defined as quality*patient satisfaction/cost) on a real time basis. This use is preferably accomplished through evaluation of groups of patient encounters for patients receiving similar treatments. Specifically, using the system 10, medical information for patients at various medical facilities may be combined, filtered and reviewed in order to test the efficacy of new drugs or treatments. By combining these large volumes of patient data from unrelated facilities, doctors are no longer required to wait for outcomes of multicenter studies in order to ascertain the effectiveness of a particular intervention, test or drug. Instead, large volumes of patient data from unrelated facilities can be combined and analyzed in order to provide doctors and the medical community with reliable treatment information. Therefore, as discussed above, this system has the advantage of reducing expenses by quickly identifying and disseminating information regarding new useful treatments as well as notifying medical personnel of failed or otherwise unacceptable treatments.

Thus disclosed is a healthcare system which provides up-to-date treatment information, and tracks the progress of the treatment and the outcome.

1. A system for assisting a practitioner in treating a patient with a medical condition, the system comprising:
a plurality of patient station interfaces, each interface having a means for entering and displaying data on a computer connected to the plurality of interfaces, the computer having a storage device for receiving information from each of the interfaces; and
a data warehouse having a plurality of templates, each template associated with a specific diagnosis, said template providing information on the treatment of the patient's condition, said template displaying said information through one of said patient interfaces.

2. The system of claim 1 wherein said data warehouse further comprises a library of medical research, and said template providing medical research from the library associated with the diagnosis.

3. The system of claim 1 wherein said template further comprises means for displaying at least one order associated with the diagnosis through the interface.

4. The system of claim 1 wherein the interface further comprises a means for prompting entry of specific patient information, the information being stored in the database.

5. A method of assisting a practitioner in the treatment of a medical condition, said method comprising:
prompting entry of patient information into a patient station interface connected to a database;
prompting entry of a diagnosis into a patient station interface;
displaying medical information associated with the diagnosis on the patient station interface; and
gathering information on the outcome of the treatment.

6. The method of claim 5 wherein after the gathering step, the method comprises analyzing the outcome and updating the medical information displayed based on the outcome.

7. The method of claim 5 wherein the displaying step further comprises filtering medical research prior to displaying the medical research.
